(19) United States
(12) Patent Application Publication
(10) Pub. No.: US 2022/0226356 A1
(43) Pub. Date: Jul. 21, 2022

(54) Composition and Concentrate for Use in Preparation of Dialysis Solution, Corresponding Dialysis Solution and Combinations Comprising Container

(71) Applicant: Fresenius Medical Care Deutschland GmbH, Bad Homburg (DE)

(72) Inventors: Tao Liu, Shanghai (CN); Runchao Lu, Shanghai (CN); Mingtao Gong, Shanghai (CN)

(21) Appl. No.: 17/609,495

(22) PCT Filed: May 10, 2019
(86) PCT No.: PCT/CN2019/086397

§ 371 Date: Nov. 8, 2021

(51) Int. Cl. A61K 31/7028; A61K 31/194; A61K 33/00; A61K 31/191; A61K 33/14; A61K 38/08; A61K 38/07; A61K 38/06; A61K 38/05; A61M 1/16

(52) U.S. Cl. CPC A61K 31/7028; A61K 31/194; A61K 33/00; A61K 31/191; A61M 1/1666; A61K 38/08; A61K 38/07; A61K 38/06; A61K 38/05; A61K 33/14

(57) ABSTRACT

Disclosed is a composition for use in preparation of a dialysis solution, at least comprising a first substance and a second substance, wherein the first substance comprises or can generate phosphate, and the second substance comprises or can generate a peptide based on glycine. Also disclosed are a corresponding dialysis solution, a corresponding concentrate, a combination of a container and the composition, a combination of a container and the dialysis solution and a combination of a container and the concentrate. According to the present disclosure, a more stable, more effective dialysis solution can be obtained.

### CROSS-REFERENCE TO RELATED APPLICATIONS

The present application is the national stage entry of International Patent Application No. PCT/CN2019/086397, filed on May 10, 2019, the disclosure of which is incorporated herein by reference.

### TECHNICAL FIELD

The present disclosure relates to a composition for use in preparation of a dialysis solution, a corresponding dialysis solution, a corresponding concentrate, a combination of a container and the composition, a combination of a container and the dialysis solution and a combination of a container and the concentrate.

### BACKGROUND ART

For treatment of a patient suffering from renal insufficiency, a dialysis process is performed to treat patients suffering from renal insufficiency. This is performed either in the peritoneum or through extracorporeal dialysis or filtration of blood. These two methods have in common the fact that dialysis fluids or dialysis solutions take up the degradation products of metabolism. These dialysis solutions usually contain bicarbonate as a buffer, an organic or inorganic acid for adjusting pH of the dialysis solution, electrolytes likes alkali metal and earth alkali metals and optionally glucose as an osmotic agent.

There are some known essential requirements, such as a sufficient stability, for the dialysis solution and various dialysis solutions have been provided to perform the dialysis process. However, the known dialysis solutions cannot meet some specific requirements, in particular for different patients. Therefore, there is a continuous need to develop more suitable dialysis solutions.

### SUMMARY OF THE DISCLOSURE

It is thus the underlying object of the present disclosure to further develop a dialysis solution and provide corresponding products.

According to a first aspect of the present disclosure, provided is a composition for use in preparation of a dialysis solution, at least comprising a first substance and a second substance, wherein the first substance comprises or can generate phosphate, and the second substance comprises or can generate a peptide based on glycine.

According to one optional embodiment of the present disclosure, the phosphate comprises at least one of phosphoric acid, sodium phosphate, disodium hydrogen phosphate, inositol phosphate, bisphosphonate, sodium dihydrogen phosphate, pyrophosphate, esters of phosphoric acid, and orthophosphate; and/or the peptide comprises at least one of glycylglycine, glycylglycylglycine, diglycine, triglycine, tetraglycine, pentaglycine, and hexaglycine.

According to one optional embodiment of the present disclosure, the inositol phosphate contains 1-6 phosphate groups; and/or the bisphosphonate is selected from a group consisted of etidronic acid, alendronic acid, risedronic acid, zoledronic acid, tiludronic acid, pamidronic acid, clodronic acid, ibandronic acid, the salts or any of the combinations thereof.

According to one optional embodiment of the present disclosure, the inositol phosphate is inositol hexaphosphate, preferably myo-inositol hexaphosphate.

According to one optional embodiment of the present disclosure, the first substance further comprises citrate; and/or the composition further comprises bicarbonate, glucose, electrolytes and osmolality formulation.

According to one optional embodiment of the present disclosure, the bicarbonate is at least partially replaced by lactate.

According to one optional embodiment of the present disclosure, the composition is provided in a solid state, preferably in a powder, granular and/or crystalline form.

According to a second aspect of the present disclosure, provided is a dialysis solution comprising the composition described in the first aspect of the present disclosure or dialysis solution components to be generated by the composition described in the first aspect of the present disclosure.

According to a third aspect of the present disclosure, provided is a concentrate for use in preparation of a dialysis solution, wherein the concentrate is allowed to be contained in one container and can generate the dialysis solution described in the second aspect of the present disclosure only by dissolving or diluting step with water.

According to one optional embodiment of the present disclosure, the concentrate is a solid concentrate at least comprising a first solid and a second solid incompatible with the first solid, which are separated from each other in the container.

According to one optional embodiment of the present disclosure, the first solid comprises bicarbonate, and the second solid comprises earth alkali metals.

According to one optional embodiment of the present disclosure, the solid concentrate further comprises a third solid compatible with both the first solid and the second solid, and the first solid, the second solid and the third solid are contained in the container in such a manner that the first solid and the second solid are separated from each other by the third solid.

According to one optional embodiment of the present disclosure, the first solid, the second solid and the third solid are in a granular form, and the first solid and the second solid are separated from each other by a layer of the third solid.

According to one optional embodiment of the present disclosure, the first solid, the second solid and the third solid are secured in place by applying a vacuum in the container, in particular configured as a flexible bag.

According to one optional embodiment of the present disclosure, the third solid is sodium chloride.

According to a fourth aspect of the present disclosure, provided is a combination of a container and the composition described in the first aspect of the present disclosure, wherein the container contains the composition.

According to a fifth aspect of the present disclosure, provided is a combination of a container and the dialysis solution described in the second aspect of the present disclosure, wherein the container contains the dialysis solution.

According to a sixth aspect of the present disclosure, provided is a combination of a container and the concentrate described in the third aspect of the present disclosure, wherein the container contains the concentrate.

### DETAILED DESCRIPTION OF PREFERRED EMBODIMENTS

Some exemplary embodiments of the present disclosure will be described hereinafter in more details to better understand the basic concept of the present disclosure.

A dialysis solution usually contains a buffer, a pH adjusting agent, electrolytes and optionally an osmotic agent, such as glucose in physiologically effective concentrations. The present disclosure is not intended to limit or specify some possible known components and concentrations thereof, as long as the dialysis solution can finally be used to treat the patient. As an example, the dialysis solution may contain one or more of sodium, potassium, calcium, magnesium, chloride, and any other suitable components customarily used in the dialysis solution. The naming of elements within the framework of the present disclosure relates to their ions.

According to a first aspect of the present disclosure, provided is a composition for use in preparation of a dialysis solution, wherein the composition at least comprises a first substance and a second substance, the first substance comprises or can generate phosphate, and the second substance comprises or can generate a peptide based on glycine.

As a usual dialysis solution, the dialysis solution often uses bicarbonate as a buffer and typically contains electrolytes, calcium and glucose in physiologically effective concentrations. In some cases, the dialysis solution further contains magnesium. However, both the calcium and magnesium ions can react with bicarbonate ion and such a reaction proceeds with the lapse of time to form insoluble fine particles or precipitates of carbonate salts, in particular at an increased pH, which can result in considerable complications in the dialysis treatment and thus is very disadvantageous.

However, according to the present disclosure, it has been found that due to the presence of phosphate, the formation of insoluble carbonate salts can be prevented even after a long period of time affording such a high pH value as 7.5 or more, even if bicarbonate ion and the calcium and magnesium ions coexist in the dialysis solution. These technical effects are entirely of unexpected nature, in particular in this case that phosphate ion also possibly can react with the calcium and/or magnesium ions to form insoluble fine particles or precipitates like bicarbonate ion. Therefore, incorporation of phosphate into the dialysis solution may achieve a more stable solution such that a dialysis solution safe in application over the complete life cycle of the product, even over a period of 24 months or longer can be ensured. Moreover, the incorporation of phosphate also results in a security on the use of the dialysis solution at a dialysis machine.

It should be pointed out at this point that the term “dialysis solution” includes any desired solution which can be used within the framework of dialysis. Concentrates are also to be understood by it which e.g. have to be further diluted or dissolved before the use in dialysis and also ready-to-use solutions which can be used as such within the framework of dialysis.

Of course, the present disclosure is not intended to limit use of any other suitable components to stabilize the dialysis solution. For example, citric acid or citrate ion can be used as a pH adjusting agent, by which the pH is adjusted to pH 7.0-7.8 to prevent the formation of insoluble fine particles or precipitates so as to provide a stable dialysis solution. The citrate (for example 1 mmol/l) has more pronounced stabilizing effect in the case of phosphate.

It is further pointed out that the term “phosphate” comprises the phosphate anion per se and also compounds which contain this ion, such as salts or esters of the phosphoric acid.

According to an exemplary embodiment of the present disclosure, the composition has an amount of phosphate such that the dialysis solution prepared from the composition contains phosphate having a concentration in a range from up to 0.4 mmol/l, preferably in a range from up to 0.375 mmol/l, or in a range from up to 0.25 mmol/l, and particularly preferably in a range from up to 0.2 mmol/l.

According to a preferable embodiment of the present disclosure, the dialysis solution contains phosphate in a range from 0.05 mmol/l to 0.25 mmol/l, in particular up to 0.20 mmol/l. The lower limit of the concentration of phosphate in the dialysis solution preferably lies at 0.05 mmol/l.

It can be found from the above that even with a low concentration of phosphate, the corresponding technical effect can be achieved. In this case, due to the fact that the phosphate concentration lies below physiological concentration values, the medical efficacy of the dialysis solution is not influenced.

As described above, according to the present disclosure, the composition also comprises the second substance which comprises or can generate peptide based on glycine. The phosphate and the peptide can achieve a synergetic effect to obtain a more stable, more effective dialysis solution.

According to an exemplary embodiment of the present disclosure, the dialysis solution contains 5-100 mmol/l of the peptide.

A peptide of this kind imparts great stability to the dialysis solution by virtue of a pH bordering on the physiological pH (7.35±0.005) endowing it with a buffering power.

In addition, this peptide has been proved to play the part of the osmotic agent, and the amount of it in the dialysis solution is accordingly inversely proportional to that of the osmotic agent such as glucose.

Also, it has been shown that a slight decrease in the bicarbonate concentration (for example to 30 mmol/l) and/or a possible increase in the peptide concentration contribute to improving the stability of the dialysis solution and are useful, in particular, for preventing a possible alkalosis in a continuous ambulatory peritoneal dialysis.

It also has been found that due to incorporation of the peptide, the pH of the dialysis solution can be maintained substantially unchanged and content of calcium, magnesium and bicarbonate ions are substantially unchanged during the storage period.

In addition, the incorporation of the peptide particularly at a higher concentration, for example 50 mmol/l can considerably increase ultrafiltration, so that a single dialysis per 24 hours (instead of four) could suffice in the case of the continuous ambulatory peritoneal dialysis.

Further, the peptide are very resistant to degradation processes and their decomposition temperature is above 270° C., which is very advantageous for heat sterilization.

It has been further found that the dialysis solution containing the peptide, which usually has a stable pH, due to the potent buffering capacity of the peptide, enhances peritoneal net ultrafiltration by maintaining a higher osmotic gradient and retarding lymphatic absorption through an increase in phosphatidylcholine concentration in the peritoneal cavity. Moreover, the dialysis solution containing the peptide has a stable pH, for example 7.35-7.40, such that the peptide absorption from the dialysis solution is slower than glucose.

Table 1 shows an exemplary embodiment of a possible composition of the dialysis solution directly for clinical use:

TABLE 1

| | Dialysis solution for clinical use |
|---|---|
| Constituent | Concentration in mmol/l |
| Sodium | 130-145 |
| Potassium | 0-3.0 |
| Calcium | 0-2.0 |
| Magnesium | 0-1.2 |
| Chloride | 90-120 |
| Hydrogen carbonate | 0-45 |
| Lactate | 0-45 |
| Phosphate | 0-0.8 |
| Glucose | 0-25 |
| Glycylglycine | 0-50 |
| pH | 7.00-7.40 |

Table 2 shows another exemplary embodiment of a possible composition of the dialysis solution:

TABLE 2

| | Dialysis solution for clinical use | |
|---|---|---|
| Constituent | Concentration in mmol/l | |
| Sodium | 131 | mmol/l |
| Calcium | 1.75 | mmol/l |
| Magnesium | 0.50 | mmol/l |
| Chloride | 101 | mmol/l |
| Bicarbonate | 20 | mmol/l |
| Lactate | 10 | mmol/l |
| Phosphate | 0.1 | mmol/l |
| Glycylglycine | 10 | mmol/l |

| | |
|---|---|
| pH | 7.35 |

The peptide can maintain the pH of the dialysis solution and the phosphate can prevent formation of the insoluble carbonate salts even if the pH increases to a value at which the insoluble carbonate salts otherwise would appear in the known dialysis solution, thereby the phosphate and the peptide cooperating with each other to allowing for achieving a particularly stable dialysis solution. Moreover, the peptide also can considerably increase ultrafiltration. Such a synergetic effect can allow for achieving a particularly stable dialysis solution simultaneously having an improved ultrafiltration capacity.

It is found that the dialysis solution (for example according to the dialysis solution according to table 2) comprising phosphate and peptide and kept in plastic bags are stable at room temperature for more than 24 months, and in particular its pH remains almost unchanged, and neither bicarbonate nor peptide are degraded. Moreover, precipitation of calcium or magnesium carbonate salts is avoided. It may be understood that the stability of pH is crucial to keep the dialysis solution stable as the precipitation occurs only at an increased pH.

For further verifying such a synergetic effect, many stability tests were performed with concentrates which need to be diluted 8-9 times, for example 8.8 times to obtain the dialysis solution.

Table 3 shows test results of comparative examples and table 4 shows test result of an example according to an exemplary embodiment of the present disclosure.

TABLE 3
Comparative examples

| | | | | Citric | | Sodium- | | Glycyl | | |
|---|---|---|---|---|---|---|---|---|---|---|
| NaCl | KCl | CaCl₂ | MgCl₂ | acid | NaHCO₃ | lactate | Phosphate | Glycine | Clear | |
| mmol/l | mmol/l | mmol/l | mmol/l | mmol/l | mmol/l | mmol/l | mmol/l | mmol/l | solution | |
| 901.3 | 17.5 | 13.1 | 4.4 | 8.8 | 204 | 102 | — | — | ~3 | h |
| 901.3 | 17.5 | 13.1 | 4.4 | 8.8 | 204 | 102 | 0.4 | — | ~4.5 | h |
| 901.3 | 17.5 | 13.1 | 4.4 | 8.8 | 204 | 102 | 0.8 | — | 6-24 | h |
| 901.3 | 17.5 | 13.1 | 4.4 | 8.8 | 204 | 102 | — | 170 | ~3 | h |
| 901.3 | 17.5 | 13.1 | 4.4 | 8.8 | 204 | 102 | — | 425 | ~2 | h |

TABLE 4
Example according to an exemplary embodiment of the present disclosure

| | | | | Citric | | Sodium- | | Glycyl | |
|---|---|---|---|---|---|---|---|---|---|
| NaCl | KCl | CaCl₂ | MgCl₂ | acid | NaHCO₃ | lactate | Phosphate | Glycine | Clear |
| mmol/l | mmol/l | mmol/l | mmol/l | mmol/l | mmol/l | mmol/l | mmol/l | mmol/l | solution |
| 901.3 | 17.5 | 13.1 | 4.4 | 8.8 | 204 | 102 | 0.4 | 85 | >24 h |
| 901.3 | 17.5 | 13.1 | 4.4 | 8.8 | 204 | 102 | 0.8 | 85 | >24 h |

As can be seen from table 3, phosphate can increase storage time, i.e. stability of the concentrates keeping clear to a certain extent and increment of the storage time increase with the concentration of phosphate. In contrast, glycylglycine cannot solely increase the storage time, even if its concentration is relatively high, for example 425 mmol/l.

But it can be seen from table 4, a combination of phosphate and glycylglycine can increase substantially the storage time such that the concentrates still keep clear after one day (24 h). Therefore, such a result comes from the synergetic effect of phosphate and glycylglycine as glycylglycine cannot solely increase the storage time as shown in table 3. Moreover, the concentration of glycylglycine is relatively low. In this case, the concentrate of phosphate also can be reduced.

It may be understood by the skilled person in the art that such a synergetic effect also occurs in the diluted solution, i.e., the dialysis solution. The stability is even more critical in a concentrated solution.

According to an exemplary embodiment of the present disclosure, the phosphate may comprise at least one of phosphoric acid, sodium phosphate, disodium hydrogen phosphate, inositol phosphate, bisphosphonate, sodium dihydrogen phosphate, pyrophosphate, esters of phosphoric acid, and orthophosphate.

According to an exemplary embodiment of the present disclosure, the peptide may comprise at least one of glycylglycine (as listed in table 1), glycylglycylglycine, diglycine, triglycine, tetragly cine, pentaglycine, and hexaglycine.

Preferably, the inositol phosphate contains 1-6 phosphate groups. The bisphosphonate may be selected from a group consisted of etidronic acid, alendronic acid, risedronic acid, zoledronic acid, tiludronic acid, pamidronic acid, clodronic acid, ibandronic acid, the salts or any of the combinations thereof.

According to an exemplary embodiment of the present disclosure, the inositol phosphate is inositol hexaphosphate, preferably myo-inositol hexaphosphate.

For further stabilizing the dialysis solution, the bicarbonate is at least partially replaced by lactate (as can be seen from table 1), which as another physiological buffer does not form precipitates with the earth alkali metals.

According to an exemplary embodiment of the present disclosure, the composition can be provided in a liquid state. However, the composition is preferably provided in a solid state, in particular in a powder, granular and/or crystalline form. The solid composition can be used to prepare the dialysis solution on site and offer the advantage of a small package volume and a low weight.

According to a second aspect of the present disclosure, provided is a dialysis solution comprising the composition described in the first aspect of the present disclosure or dialysis solution components to be generated by the composition described in the first aspect of the present disclosure.

According to a third aspect of the present disclosure, provided is a concentrate for use in preparation of a dialysis solution, wherein the concentrate is allowed to be contained in one container and can generate the dialysis solution described in the second aspect of the present disclosure only by dissolving or diluting step with water. In this case, the concentrate comprises all components except for water in one compartment with a sufficient stability and the dialysis solution prepared by the concentrate also has a sufficient stability.

According to an exemplary embodiment of the present disclosure, the concentrate is a solid concentrate at least comprising a first solid and a second solid incompatible with the first solid, which are separated from each other in the container. Compatible here is defined as not causing any mutual changes in their chemical and/or physical properties whereas incompatible or not compatible is defined conversely.

According to an exemplary embodiment of the present disclosure, the first solid comprises bicarbonate, and the second solid comprises earth alkali metals.

According to an exemplary embodiment of the present disclosure, the solid concentrate further comprises a third solid compatible with both the first solid and the second solid, and the first solid, the second solid and the third solid are contained in the container in such a manner that the first solid and the second solid are separated from each other by the third solid.

According to an exemplary embodiment of the present disclosure, the first solid, the second solid and the third solid are in a granular form, and the first solid and the second solid are separated from each other by a layer of the third solid.

According to an exemplary embodiment of the present disclosure, the first solid, the second solid and the third solid are secured in place by applying a vacuum in the container, in particular configured as a flexible bag. In this case, the first solid, the second solid and the third solid can be pressed by the container.

According to an exemplary embodiment of the present disclosure, the third solid is sodium chloride.

According to a fourth aspect of the present disclosure, provided is a combination of a container and the composition described in the first aspect of the present disclosure, wherein the container contains the composition.

According to a fifth aspect of the present disclosure, provided is a combination of a container and the dialysis solution described in the second aspect of the present disclosure, wherein the container contains the dialysis solution.

According to a sixth aspect of the present disclosure, provided is a combination of a container and the concentrate described in the third aspect of the present disclosure, wherein the container contains the concentrate.

While certain embodiments have been described, these embodiments have been presented by way of example only, and are not intended to limit the scope of the present disclosure. The attached claims and their equivalents are intended to cover all the modifications, substitutions and changes as would fall within the scope and spirit of the present disclosure.

1-18. (canceled)

19. A composition for use in preparation of a dialysis solution, the composition comprising a first substance and a second substance, wherein the first substance comprises or can generate phosphate, and the second substance comprises or can generate a peptide based on glycine.

20. The composition according to claim 19, wherein:
the phosphate comprises at least one of: phosphoric acid, sodium phosphate, disodium hydrogen phosphate, inositol phosphate, bisphosphonate, sodium dihydrogen phosphate, pyrophosphate, esters of phosphoric acid, and orthophosphate; and/or
the peptide comprises at least one of: glycylglycine, glycylglycylglycine, diglycine, triglycine, tetraglycine, pentaglycine, and hexaglycine.

21. The composition according to claim 20, wherein:
the inositol phosphate contains 1-6 phosphate groups; and/or
the bisphosphonate is selected from a group consisting of: etidronic acid, alendronic acid, risedronic acid, zoledronic acid, tiludronic acid, pamidronic acid, clodronic acid, ibandronic acid, and the salts or any of the combinations thereof.

22. The composition according to claim 21, wherein the inositol phosphate is inositol hexaphosphate, preferably myo-inositol hexaphosphate.

23. The composition according to claim 19, wherein:
the first substance further comprises citrate; and/or
the composition further comprises bicarbonate, glucose, electrolytes and osmolality formulation.

24. The composition according to claim 20, wherein:
the first substance further comprises citrate; and/or
the composition further comprises bicarbonate, glucose, electrolytes and osmolality formulation.

25. The composition according to claim 21, wherein:
the first substance further comprises citrate; and/or
the composition further comprises bicarbonate, glucose, electrolytes and osmolality formulation.

26. The composition according to claim 23, wherein the bicarbonate is at least partially replaced by lactate.

27. The composition according to claim 19, wherein the composition is provided in a solid state, preferably in a powder, granular and/or crystalline form.

28. A dialysis solution comprising the composition according to claim 19 or dialysis solution components to be generated by the composition according to claim 19.

29. A concentrate for use in preparation of a dialysis solution, wherein the concentrate is allowed to be contained in one container and can generate the dialysis solution according to claim 28 only by dissolving or diluting step with water.

30. The concentrate according to claim 29, wherein the concentrate is a solid concentrate at least comprising a first solid and a second solid incompatible with the first solid, which are separated from each other in the container.

31. The concentrate according to claim 30, wherein the first solid comprises bicarbonate, and the second solid comprises earth alkali metals.

32. The concentrate according to claim 30, wherein:
the solid concentrate further comprises a third solid compatible with both the first solid and the second solid, and
the first solid, the second solid and the third solid are contained in the container in such a manner that the first solid and the second solid are separated from each other by the third solid.

33. The concentrate according to claim 31, wherein:
the solid concentrate further comprises a third solid compatible with both the first solid and the second solid, and
the first solid, the second solid and the third solid are contained in the container in such a manner that the first solid and the second solid are separated from each other by the third solid.

34. The concentrate according to claim 32, wherein:
the first solid, the second solid and the third solid are in a granular form, and
the first solid and the second solid are separated from each other by a layer of the third solid.

35. The concentrate according to claim 34, wherein the first solid, the second solid and the third solid are secured in place by applying a vacuum in the container, in particular configured as a flexible bag.

36. The concentrate according to claim 32, wherein the third solid is sodium chloride.

37. The concentrate according to claim 34, wherein the third solid is sodium chloride.

38. The concentrate according to claim 35, wherein the third solid is sodium chloride.
